(19) United States
(12) Patent Application Publication
(10) Pub. No.: US 2023/0233362 A1
(43) Pub. Date: Jul. 27, 2023

(54) DEVICE AND METHOD FOR REVERSIBLE OCCLUSION OF BODY LUMENS

(71) Applicants: Yissum Research Development Company of the Hebrew University of Jerusalem Ltd, Jerusalem (IL); Hadasit Medical Research Services & Development Limited, Jerusalem (IL)

(72) Inventors: Daniel COHN, Jerusalem (IL); Allen I BLOOM, Neve Ilan (IL)

(21) Appl. No.: 18/159,185

(22) Filed: Jan. 25, 2023

Related U.S. Application Data
(60) Provisional application No. 63/267,114, filed on Jan. 25, 2022.

(51) Int. Cl. A61F 6/22

(52) U.S. Cl. CPC A61F 6/22; A61F 2210/0023

(57) ABSTRACT

The technology disclosed herein generally concerns a system and method for reversibly occluding a body lumen.

### FIELD OF THE INVENTION

The present invention generally pertains to the reversible occlusion of bodily lumens and to methods thereof.

### BACKGROUND OF THE INVENTION

Hydrogels are considered one of the leading types of biomaterials, since they combine several advantageous attributes, among them their enhanced biocompatibility, the minimal inflammatory response and thrombosis they elicit, and the fact that they do not cause any tissue damage. Additionally, hydrogels are characterized by high permeability levels of oxygen, small nutrients and other low molecular weight water soluble metabolites, and they also display tunable fluid absorbance levels and superior transparency.

Even though not of universal applicability, in situ generated hydrogels are advantageous when compared to pre-formed ones, most importantly due to their high conformability to the shape of the luminal cavity, regardless of its geometric complexity. Their ease of deployment, their universal adaptability and the comfort they offer the patient are additional advantageous features of in situ formed hydrogels. Moreover, since the administration of in situ formed displaying hydrogel occlusion devices is rapid, clean, hands free and straightforward, higher patient levels of compliance are achieved.

In light of their theoretical advantageous features, as enumerated above, the small number of in situ generated occlusion devices currently in the clinic, is surprising. The most important problems of the in situ generated occlusion devices investigated to date, including those currently in the clinic, pertain to the fact that their vast majority convert the liquid into a gel via concentrated polymerization or crosslinking reactions. The drawbacks of this approach stem primarily from the fact that the reactions used are typically unacceptably slow for this application. Even more importantly, these reactions entail the use of hazardous toxic compounds. Additional drawbacks of these occlusion devices stem from the fact that in most cases they are not user friendly and cumbersome to form, since most of them require mixing two components to for effective administration. Furthermore, having to mix two components may make the devices also not homogeneous and not reproducible.

Removal of implants or occlusion devices, after a period of intimate contact with a surrounding tissue and their secretions and the development of tissue adhesions is almost unavoidable, not only extremely painful but also, and most importantly, results in tearing and injuring the tissues, and triggering an additional detrimental tissue response. In some instances, these processes are life threatening.

### SUMMARY OF THE INVENTION

The present invention discloses a new type of occlusion device displaying advantageous features, which are at least partially derived from presence of at least one environmentally responsive component, that is capable of reversion between two states: a high viscosity state at physiological conditions, namely under the conditions of the site of performance, and a low viscosity state that allows for deployment and removal from the tissue or organ lumen upon application of an environmental stimulus (e.g., temperature, pH, ionic strength or light of various wavelengths, and others).

Contrary to the prior art, the invention disclosed herein concerns novel occlusion devices for deployment in the luminal cavity of, e.g., the fallopian tubes or vas deferens, that comprise RTR and in some instances also non-RTR materials. By selecting and controlling the chemical and mechanical properties of the RTR material(s) and the non-RTR material(s), where present, as well as the ratio between them, the different properties of the occlusion device can be fine-tuned.

The fundamental feature of the invention common to all aspects, compositions and utilities of the invention is the in situ generation of a stimulus-responsive bio-durable occlusion device that makes the deployment and removal of said device efficacious, devoid of pain and minimally disruptive to the tissues involved.

Thus, in its most general aspect, the invention concerns an occlusion material performing as an occlusion device, which comprises a stimulus-responsive bio-durable material exhibiting a solid or semi-solid state at a physiological temperature (i.e., body temperature) and a liquified state at a temperature lower than the physiological temperature. Occlusion devices and occlusion materials used according to the invention are not crosslinked materials. Occlusion devices and occlusion materials used according to the invention are free of any crosslinking bonds between building blocks or molecules making up the stimulus-responsive materials.

As used herein, the term “occlusion device” encompasses any of the occlusion materials disclosed herein that are capable of solidifying under physiological conditions (temperature, pH, humidity, ionic strength, etc) and liquifying at non-physiological conditions. The term ‘occlusion device’ is therefore interchangeable with ‘occlusion material’.

In a first aspect, there is provided a temporary occlusion device configured to or capable of solidify and occlude a bodily lumen under physiological conditions (temperature, pH, humidity, ionic strength, etc) and liquefy at non-physiological conditions.

As used herein, the term “temporary” reflects the residence time of the occlusion device in the body lumen and the fact that the device may be removed therefrom upon demand or at any time following its instillation, as further described herein.

Also provided is a stimulus-responsive occlusion material, the material exhibiting a solid or semi-solid state at a physiological temperature (i.e., body temperature) and a liquified state at a temperature lower than the physiological temperature, wherein the material is a single material or a mixture of materials provided in neat, solution, suspension or gel form, or a composition or a solution or a suspension comprising a carrier such as water.

In some embodiments, the occlusion material or occlusion device is or comprises at least one reverse thermo-responsive (RTR) polymer or a water-formulation comprising such a polymer. In other words, in an occlusion device of the invention, the stimulus-responsive occlusion material (single material or a combination of materials) exhibits a solid or semi-solid state at a physiological temperature (i.e., body temperature being around 37° C.) and a liquified state at a temperature lower than the physiological temperature. Putting it differently, the reverse thermo-responsive (RTR) polymer is one that generates aqueous solutions that display low viscosities at low temperatures, below or at a body temperature, and exhibit a sharp viscosity increase as the temperature rises within a narrow temperature interval, producing a solid or semi-solid gel at the body temperature.

As the occlusion material may revert from a solid state to a liquified state under conditions of external or environmental stimuli, temporary occlusion may be achievable, whereby the solid or semi-solid occluder is caused to undergo partial or complete liquification which permits non-injurious reopening of the occluded lumen. Thus, the terms “temporary occluding device”, a “reversible occluding device” and a “revocable occluding device” are used interchangeably to encompass any device of the invention that transitorily occludes a bodily lumen, which is open during its normal physiological function. The “device” used for achieving occlusion is typically an unshaped material composition or formulation or a solution or a gel or otherwise a material form, which is configured for delivery into a lumen to be occluded, such that it can flow into the lumen and fill the lumen to an extent that prevents transport or transition of liquids, solids, cells or tissue material through the lumen. The term “device” should therefore be understood in its broadest meaning to encompass any such material form. In some embodiments, for certain applications, the device, namely the composition/formulation or occluding polymer as such, may be provided on a substrate. In some embodiments, said substrate is a non-RTR material, such as a polymer or a particulate material or any other material. In other embodiments, said composition/formulation provided on a substrate is combined with RTR polymers of the invention.

In some embodiments, the occlusion device is in a form of a polymer or a polymeric system which is thermo-sensitive. In other words, as defined, the polymer or polymeric system achieves significant chemical, mechanical, physical and/or biological changes, due to small temperature differentials. The resulting change is based on different mechanisms such as ionization or entropy-gain due to, e.g., water structured molecules release, among others.

The device comprises an RTR displaying polymer(s) that gels and generates an occluding device upon deployment, as the polymer(s) comes in contact with a tissue. The in situ generated RTR displaying occluding device may comprise also additional materials, having different configurations, for different purposes, such as, without limitation, to optimize its viscosity, to fine tune its transport properties, to enhance the occluding device attachment to the luminal tissues it comes in contact with, and to enhance the ability of the occluding device to retain its water content. Thus, in some embodiments, the device comprises one or more additional non-RTR material(s).

In some embodiments, the RTR polymer used in occlusion devices of the invention comprises poly(ethylene oxide) (PEO), poly(propylene oxide) (PPO) or mixtures thereof. In some embodiments, the RTR comprises both PEO and PPO.

In some embodiments, RTR polymer may comprise chain extended PPO and/or PEO units, or diblocks or triblocks thereof.

In some embodiments, each of the building blocks, e.g., PEO and PPO may be associated via chain extending or linker functionalities, such as bifunctional materials, to afford the RTR polymer. Where E designates a chain extender moiety or a linker, RTR polymers of the invention may comprise blocks or segments of the form, e.g., -E-PPO-, -E-PPO-PEO, -PPO-E-PEO-, -PPO-PEO-E-PPO-, etc as well as diblocks and triblocks thereof; moiety E being as defined hereinbelow.

The distribution of the PPO and/or PEO in the RTR polymer may vary or may be specifically tailored to exhibit specific properties. In some embodiments, the PPO and PEO are arranged in diblocks of PPO-PEO or triblocks of PPO-PEO-PPO or PEO-PPO-PEO, wherein the number of PPO and/or PEO in each diblock or triblock may vary. For example, a diblock of PEO and PPO may be of the form PEOn-PPOm, wherein each of n and m are integers between 1 and 200. Similarly, in a triblock PEO-PPO-PEO, the number of PEO and PPO may vary, such that in a triblock PEOn-PPOm-PEOn, each of n and m is between 1 and 200.

In some embodiments, the RTR polymer used in occlusion devices of the invention is or comprises.
poly(ethylene oxide)-poly(propylene oxide)-poly(ethylene oxide) (PEO-PPO-PEO) triblocks;
random or alternating reverse thermo-responsive PEO-PPO block copolymers such as those described in US Patent Application No. US 2003/0082235, herein incorporated by reference;
N-alkyl substituted acrylamides (such as poly-N-isopropyl acrylamide [PNIPAAm]);
cellulose derivatives selected from hydroxypropyl methylcellulose and hydroxypropyl cellulose, poly(ethylene oxide)-polylactic acid copolymers;
poly(ethylene oxide)-polycaprolactone copolymers; and
various amphiphilic polymers such as poly(ethylene oxide)-polylactic acid block copolymers.

In some embodiments, the RTR polymer or mixture of polymers is or comprises a polymer having or comprising the triblock poly(ethylene oxide)/poly(propylene oxide)/poly(ethylene oxide) (PEO-PPO-PEO). In some embodiments, the RTR polymer or mixture of polymers comprises poly(ethylene oxide) and poly(propylene oxide) blocks connected together via a bi-functional material, e.g., acting as a chain extender molecule or a linker molecule.

The invention thus provides a stimulus-responsive occlusion material, the material exhibiting a solid state at a physiological temperature (i.e., body temperature) and a liquified state at a temperature lower than the physiological temperature, said material being a polymer or a polymeric mixture of polymers comprising poly(ethylene oxide)/poly(propylene oxide)/poly(ethylene oxide) (PEO-PPO-PEO) triblock.

In some embodiments, the occlusion material is or comprises a polymer of the structure

-[E-(BCB)]n-

wherein

B is polyethylene oxide (PEO),

C is polypropylene oxide (PPO),

n is an integer designating the number of blocks in the polymer; n being 2 or more, and

E is a group or a chain extender moiety connecting any triblock to another triblock.

In some embodiments, the occlusion material is a polymer of the structure

-E-(BCB)-E-(BCB)-E-(BCB) . . .

In RTR polymers of the invention, the termini may be substituted with end groups such as H, polyethylene glycol (PEG) of various molecular weights or polypropylene glycol (PPG) of various molecular weights, as further described herein.

The unit (BCB) designates the triblock -(PEO)-(PPO)-(PEO)-, wherein the PEO and PPO are covalently bonded to each other to adopt the triblock structure:

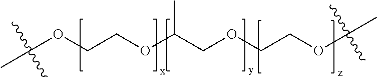

(or as depicted for example in FIG. 1), wherein each of the dashed lines designated bond of association with another triblock or a deblock or PEO or PPO, or with a linker or chain extender moiety or with a terminal group such as H atom, PEG or PPG, as further disclosed herein, and wherein x, y and z define the size of the PEO (integer x and integer z) and PPO (integer y) segments, namely they are each an integer designating the number of EO units (integers x and z) and PO units (integer y) in the triblock.

In some embodiments, x and z, each designating a number of EO units in a PEO segment, may independently be between 1 and 200. Integer y, defining the number of PO units in a PPO segment, may be between 10 and 100.

In some embodiments, in a polymer used according to the invention, having the structure -[E-(BCB)]n, as defined herein, n may be between 2 and 80, or between 2 and 40 or between 2 and 10, or between 2 and 4. In other words, the polymer may have the structure -[E-(BCB)]₂ (being -E-(BCB)-E-(BCB)-) or any structure having up to n=80 (namely -[E-(BCB)]₈₀), wherein each of the triblock (BCB) is -(PEO)-(PPO)-(PEO)-, or as defined above. The termini groups of the polymer may be H, PEG or PPG, as defined herein.

In some embodiments, in a triblock of the structure

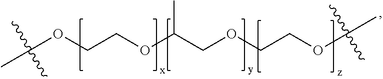

variables x, y and z may be as indicated in Table 1:

TABLE 1
Exemplary triblock units

| Triblock | X | y | z |
|---|---|---|---|
| 1 | 99 | 65 | 99 |
| 2 | 60 | 39 | 60 |
| 3 | 54 | 73 | 54 |
| 4 | 148 | 56 | 148 |
| 5 | 125 | 47 | 125 |
| 6 | 102 | 39 | 102 |
| 7 | 80 | 30 | 80 |
| 8 | 37 | 56 | 37 |
| 9 | 26 | 39 | 26 |
| 10 | 23 | 73 | 23 |
| 11 | 20 | 30 | 20 |
| 12 | 11 | 16 | 11 |
| 13 | 25 | 56 | 25 |
| 14 | 21 | 47 | 21 |
| 15 | 17 | 39 | 17 |
| 16 | 13 | 30 | 13 |
| 17 | 9 | 21 | 9 |
| 18 | 9 | 30 | 9 |
| 19 | 5 | 30 | 5 |
| 20 | 2 | 16 | 2 |

In some embodiments, in a triblock of the structure above, each of x and z is independently an integer between 2 and 150. In some embodiments, each of x and z is an integer between 2 and 20, or 20 and 30, or 31 and 100 or between 101 and 150.

In some embodiments, each of x and z is independently an integer selected from 2, 5, 9, 11, 13, 20, 21, 23, 25, 26, 37, 54, 60, 80, 99, 102, 125, and 148.

In some embodiments, x and z are same. In some embodiments, x and z are different.

In some embodiments, in a triblock of the structure above, y is between 10 and 100, or between 10 and 90, 10 and 80, 10 and 70, 10 and 60, 15 and 75, 20 and 75, 30 and 75, 40 and 75, 50 and 75, 30 and 65, 40 and 75 or 50 and 75.

In some embodiments, y is 16, 21, 30, 39, 47, 56, 65, or 73.

In some embodiments, the triblock is any one or more of:

-PEO₉₉-PPO₆₅-PEO₉₉,

-PEO₆₀-PPO₃₉-PEO₆₀,

-PEO₅₄-PPO₇₃-PEO₅₄,

-PEO₁₄₈-PPO₅₆-PEO₁₄₈,

-PEO₁₂₅-PPO₄₇-PEO₁₂₅,

-PEO₁₀₂-PPO₃₉-PEO₁₀₂,

-PEO₈₀-PPO₀₃-PEO₈₀,

-PEO₃₇-PPO₅₆-PEO₃₇,

-PEO₂₆-PPO₃₉-PEO₂₆,

-PEO₂₃-PPO₇₃-PEO₂₃,

-PEO₂₀-PPO₃₀-PEO₂₀,

-PEO₁₁-PPO₁₆-PEO₁₁,

-PEO₂₅-PPO₅₆-PEO₂₅,

-PEO₂₁-PPO₄₇-PEO₂₁,

-PEO₁₇-PPO₃₉-PEO₁₇,

-PEO₁₃-PPO₃₀-PEO₁₃,

-PEO₉-PPO₂₁-PEO₉,

-PEO₉-PPO₃₀-PEO₉,

-PEO₅-PPO₃₀-PEO₅,

-PEO₂-PPO₁₆-PEO₂.

In some embodiments, an RTR polymer used according to the invention comprises a triblock selected from:

-PEO₉₉-PPO₆₅-PEO₉₉,

-PEO₆₀-PPO₃₉-PEO₆₀,

-PEO₅₄-PPO₇₃-PEO₅₄,

-PEO₁₄₈-PPO₅₆-PEO₁₄₈,

-PEO₂₅-PPO₄₇-PEO₁₂₅,

-PEO₁₀₂-PPO₃₉-PEO₁₀₂,

-PEO₈₀-PPO₃₀-PEO₈₀,

-PEO₃₇-PPO₅₆-PEO₃₇,

-PEO₂₆-PPO₃₉-PEO₂₆,

-PEO₂₃-PPO₇₃-PEO₂₃,

-PEO₂₀-PPO₃₀-PEO₂₀,

-PEO₁₁-PPO₁₆-PEO₁₁,

-PEO₂₅-PPO₅₆-PEO₂₅,

-PEO₂₁-PPO₄₇-PEO₂₀,

-PEO₁₇-PPO₃₉-PEO₁₇,

-PEO₁₃-PPO₃₀-PEO₁₃,

-PEO₉-PPO₂₁-PEO₉,

-PEO₉-PPO₃₀-PEO₉,

-PEO₅-PPO₃₀-PEO₅,

-PEO₂-PPO₁₆-PEO₂.

In some embodiments, the triblock may have a molecular weight between 1,050 and 16,250 Da. In some embodiments, the triblock may have a molecular weight selected from 12,600, 7,500, 6,830, 16,250, 13,750, 11,250, 8,750, 6,500, 4,500, 4,100, 3,500, 1, 900, 5,420, 4,580, 3,750, 2,920, 2,000, 2,500, 2,190 and 1,050 Da.

In some embodiments, the triblock may have between 10 and 80 wt % PEO content.

In some embodiments, the triblock may be selected from:

-PEO₉₉-PPO₆₅-PEO₉₉, having 70% wt PEO content;

-PEO₆₀-PPO₃₉-PEO₆₀, having 70% wt PEO content

-PEO₅₄-PPO₇₃-PEO₅₄, having 70% wt PEO content

-PEO₁₄₈-PPO₅₆-PEO₁₄₈, having 80% wt PEO content

-PEO₁₂₅-PPO₄₇-PEO₁₂₅, having 80% wt PEO content

-PEO₁₀₂-PPO₃₉-PEO₁₀₂, having 80% wt PEO content

-PEO₈₀-PPO₃₀-PEO₈₀, having 80% wt PEO content

-PEO₃₇-PPO₅₆-PEO₃₇, having 50% wt PEO content

-PEO₂₆-PPO₃₉-PEO₂₆, having 50% wt PEO content

-PEO₂₃-PPO₇₃-PEO₂₃, having 50% wt PEO content

-PEO₂₀-PPO₃₀-PEO₂₀, having 50% wt PEO content

-PEO₁₁-PPO₁₆-PEO₁₁, having 50% wt PEO content

-PEO₂₅-PPO₅₆-PEO₂₅, having 40% wt PEO content

-PEO₂₁-PPO₄₇-PEO₂₁, having 40% wt PEO content

-PEO₁₇-PPO₃₉-PEO₁₇, having 40% wt PEO content

-PEO₁₃-PPO₃₀-PEO₁₃, having 40% wt PEO content

-PEO₉-PPO₂₁-PEO₉, having 40% wt PEO content

-PEO₉-PPO₃₀-PEO₉, having 30% wt PEO content

-PEO₅-PPO₃₀-PEO₅, having 20% wt PEO content

-PEO₂-PPO₁₆-PEO₂, having 10% wt PEO content.

The linker moiety or chain extender moiety E is a group that covalently bridges and thus associates any two triblock units to each other, or any PPO or PEO or diblocks thereof to each other or to another, as defined, and is typically derived from a linker or a chain extender molecule or a material that is reactive with triblock units' reactive ends. Each of the units, e.g., triblock units, to be associated may have terminal reactive OH groups, COOH groups, amine groups, aldehyde groups or thiol groups. Thus, depending on the end groups of the triblock units and the functionalities of the linker or chain extender material, the nature of functionality that associates the units and the linker may differ. For example, a terminal hydroxy group reacted with an aliphatic diisocyanate will result in a urethane group. Where the reactive terminal groups are COOH groups, amides may be formed and in the case of terminal amine groups, urea groups may be formed. By selecting the type of linker or chain extender material to be used, the reactive terminal groups of the triblock units, as well as other features of the triblock units, the structure of the extender moiety E may be tailored to modify the mechanical and rheological properties of the resulting polymers, to achieve robust materials displaying enhanced properties such as biodurability.

In some embodiments, the RTR polymer comprises functionalized or end-capped PPO, PEO or triblocks thereof, permitting chain extension. In such configurations, for example, equimolar mixture of C═C end-capped triblocks and amino- or thiol-terminated triblocks, are provided under Michael addition reaction conditions to chain extend the triblocks.

To associate two same or different triblock units, each having same or different terminal reactive groups, e.g., OH, COOH, amines, etc, the linker or chain extender material used for associating the triblock units may be selected amongst bifunctional materials capable of reacting with the terminal groups of the PEOs in each BCB triblock. The bifunctional material comprises two functionalities that each is capable of reacting with a terminal group of a unit, e.g., a triblock unit, so as to associate or to form a bridge therebetween. Typically, the bifunctional material does not contain any other reactive functionality that can react with the triblock or with another of the functional materials.

In some embodiments, the linker or chain extender molecule may be selected amongst any bifunctional materials capable of forming two covalent bonds, each covalent bond with a different terminal atom or group of a triblock unit.

In some embodiments, the linker or chain extender molecule may be selected amongst any bifunctional materials capable of forming covalent bonds with hydroxy oxygen atoms of the PEO units. The bifunctional materials may have carboxylic acid functionalities, halide functionalities, anhydride functionalities, acyl functionalities, isocyanate functionalities, cyanate functionalities, ester functionalities, sulfonyl functionalities, and others. The linker or chain extender molecule may be a bifunctional material having two identical functionalities reactive with the terminal reactive groups of the PEO units, e.g., hydroxy groups, or two different functionalities, each having a reactivity towards a terminal reactive group of a PEO unit.

In other words, the linker or chain extender molecule may be a material of the form X—R—X1, wherein each of X and X1, being same or different, is selected from a functionality capable of reacting with an end group or end atom of the units to be associated, e.g., triblock units, and R is a spacer which length and composition may be varied. In some embodiments, each of X and X1 may be selected from anhydrides, acyls, isocyanates, cyanates, esters, sulfonyls, carboxylic acids, hydroxy groups, etc; namely, the linker molecule of the structure X—R—X1 may be a bifunctional molecule as defined herein.

In some embodiments, each of the triblock units comprises PEO units with hydroxy terminal groups. In some embodiments, the linker or chain extender material is selected from di-isocyanates, di-acyl chlorides, di-carboxylic acids and di-anhydrides. In some embodiments, the linker or chain extender molecule is a di-isocyanate, e.g., having the generic structure:

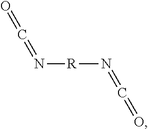

wherein the R may vary as known in the art. For example, R may be an aliphatic moiety having between 1 and 12 methylene groups, branched or linear, may be an aromatic group, may be an aralkyl group (comprising both aryl functionalities and alkylene functionalities), etc. Irrespective of the length and composition of R, a product of the reaction between a di-isocyanate chain extender molecule and hydroxy oxygen atoms of the PEO is a moiety E of the structure

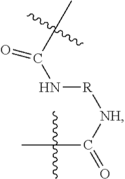

wherein the dashed lines indicate bonds formed with the oxygen atoms of the PEO units. The bonds formed are urethane bonds.

As the nature of variant R may be varied, the bifunctional material enables both extension and association of any two neighboring triblock units, thereby permitting further modification of properties of the polymer, to enable reversible occlusion, as discussed herein.

Thus, the chain extender moiety E is derived from the linker or chain extender molecule that is used. In some embodiments, moiety E is derived from a di-isocyanate having a variable R selected amongst alkylene moieties or alkylene comprising moieties. The alkylene may comprise between 2 and 12 carbon atoms, in a linear or branched structure.

In some embodiments, the moiety E is derived from an alkyl di-isocyanate, having between 2 and 12 carbon atoms.

In some embodiments, the moiety E is derived from an aromatic di-isocyanate, wherein the aromatic moiety of the aromatic di-isocyanate having between 6 and 10 carbon atoms.

In some embodiments, the moiety E is derived from an aralkyl di-isocyanate, having between 7 and 20 carbon atoms, e.g., wherein the aralkyl comprises an aryl group, such as a phenyl, and at least one exo-cyclic carbon group such as methylene.

In some embodiments, the moiety E is derived from a di-isocyanate selected from hexamethylene di-isocyanate (HDI), methylene di-phenyl di-isocyanate (MDI), isophorone diisocyanate, toluene di-isocyanate and any other di-isocyanate. In other words, in such examples, R is hexamethylene (in case of HDI), diphenyl methylene (in case of MDI), isophorone or toluene.

In some embodiments, the di-isocyanate is HDI. In such a case, E is of the structure

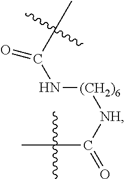

wherein the dashed lines indicate bonds formed with the oxygen atoms of the PEO units.

In some embodiments, the linker or chain extender material may be selected amongst di-isocyanates such as hexamethylene di-isocyanate (HDI), methylene di-phenyl di-isocyanate (MDI), H-MDI, toluene di-isocyanate (TDI), L-lysine ethyl ester diisocyanate (LDI), IDI and others.

In some embodiments, the linker or chain extender material may be selected amongst acyl chlorides, oxaloyl, succinoyl, glutaroyl, adipoyl, pimeloyl, suberoyl, therphthaloyl, isophthaloyl, phthaloyl, phosgene and others.

In some embodiments, the chain extended polymers of the invention may have polymer end groups such as PEG or PPG. The various PEG and PPG groups may be selected from PEG1000, PEG2000, PEG3000, PGE3400, PEG4000, PEG6000, PEG8000, PPG1000, PPG2000, PPG300, PPG4000 and PEG or PPG of other molecular weights.

The chain extended RTR polymers used herein are not crosslinked systems. These materials are thermoplastic materials so that the key requirement of “on command” liquefiability (fully or partially) is retained. In some configurations, to achieve effective luminal delivery and subsequent occlusion, a device of the invention may comprise more than one RTR polymer, optionally in combination with one or more non-RTR components. The non-RTR component may be any material, including a polymeric material, a drug, a salt that in water can affect the ionic strength of the system, micro- or nanoparticles or nanofibers, magnetic nanoparticles and others. In some configurations, the non-RTR component is a biodegradable polymer.

In some configurations, the RTR component is biodegradable, comprising biodegradable moieties such as lactide, glycolide or caprolactone units, among others.

The RTR polymer and the non-RTR component may have the same or a different composition and/or concentration and/or molecular weight.

Devices and compositions disclosed herein may also comprise components that are responsive to different environmental stimuli or may perform other tasks, such as, without limitation, other chemical, physical, mechanical or biological functions and combinations thereof. The composition and properties of the RTR and/or non-RTR component(s) may also vary along any of the axes of the occluding device, having programmed properties at different points of the occluding device.

Where a non-RTR component is present, the RTR and the non-RTR may be configured or formulated to be administered simultaneously or sequentially.

In some configurations, one or more RTR polymers may be associated to each other and/or to a non-RTR component(s) so as to form an integral part of the occluding device. The association between the components may be covalent, ionic, physical entrapment or any other type of binding. In some embodiments, the RTR polymer(s) may form, without limitation, an RTR layer. In some embodiments, the layer may comprise, without limitation, partially or exclusively, RTR-displaying chains attached, covalently or otherwise, to a surface of a suitable substrate. Typically, the RTR component is deployed at a temperature below the relevant thermal transition and will gel, partially or totally, sharply or gradually, as the system heats up to the temperature of the site. Towards removal, the occluding device is cooled down, below the relevant thermal transition of the RTR gel, substantially weakening the interfacial layer and, typically, liquefying it (partially or fully), making the detachment of the occluding device from the luminal site and/or of the surrounding tissues easy, not painful, as well as not injurious to the luminal tissue.

The RTR polymer may be provided in a form of solid particles of any geometry, hollow or solid, that comprise the RTR polymer. The particles may be non-RTR solid particles, in which the RTR is isotropically or anisotropically, homogeneously or non-homogenously distributed. The particles may be nanometric, micrometric, millimetric or macroscopic in size. The particles may dissolve in the aqueous solution over time.

The RTR polymers may be fully or partially attached or grafted to a surface region of a solid substrate, such as micro- or nanoparticles. At low temperatures, below the transition temperature, the chains of the RTR polymers not attached to the solid substrate and those grafted onto the surface of the particles are uncoiled and stretched out. When the device is deployed, as it heats up the chains coil and become entangled. The particles being connected non-covalently and reversibly with the RTR component enhance the mechanical properties of the device and its long-term in vivo stability, and prevent passage of tissues or cells or any material therethrough. Grafting may be achievable by reacting the RTR chains or their precursors to functional groups present on the solid substrate surface layer or on the surface of any of the components of the device, or by generating reactive anchoring sites on the surface of any of them by various techniques such as, without limitation, e-beam, UV or gamma radiation, chemical reactions and plasma treatments. This can be exemplified, without limitation, for plasma treatments, by exposing the occluding device for example to plasma of ammonia, whereby amine moieties are generated on the surface of the solid substrate of the occluding device. These reactive groups perform then as anchoring sites for the RTR chains to bind to directly or via a coupling agent. Among other reactants, the substrate can be exposed to plasma of air, whereby various reactive moieties, such as OH and COOH groups, are formed on the surface exposed that will perform as anchoring sites for the RTR chains.

An occluding device according to the invention may comprise one or more additional solid components that can be provided in a diversity of shapes, sizes and geometries, including, without limitation, spheres, particles of any other shape, capsules, fibers, ribbons, films, meshes, fabrics, non-woven structures, foams, porous structures of different types, each having the possibility of being solid, porous, hollow and/or combinations thereof. These component(s) may or may not be associated to the RTR polymer.

In some configurations, the additional components may be solid components, such as solid polymeric components, already at the time of deployment or may be generated and/or solidified in situ during or immediately after deployment or over time. The solid components may differ significantly, without limitation, in their composition, behavior and in their different properties. They may be present as solids for the whole period during which the occluding device is at the luminal site, or they may change their composition over time and/or may degrade and/or swell and/or dissolve and/or crystallize, and combinations thereof.

Occlusion devices of the invention may be generally provided free of any drug or active material or may be provided or administered loaded with a pharmaceutically active substance. Such materials may be selected, without limitation, from drugs and drug residues, oligopeptide sequences, growth factors, hormones, materials containing genetic information, cells, contraceptive agents, ion eluting agents, metals or metallic materials, e.g., copper which may be used as contraceptive, anti-restenosis agents, antibacterial agents, antifungal agents, antimicrobial agents, antibiotics, and others. The pharmaceutically acceptable agents, being of pharmacological and/or biological relevance, may be blended with any of the device component(s), prior to, during or after deployment, and/or may be attached, covalently or otherwise, to one or more of the occluding device components, e.g., the RTR and/or non-RTR components.

In some embodiments, the active materials are encapsulated or carried in a solid vehicle such as nanocarriers and microcarriers or in a liposome or in any micellar structure.

In some embodiments, the device comprises at least one material capable of promoting regeneration of a healthy tissue at the occlusion site and or an agent to inhibit formation of scar tissue and fibrosis at the occlusion site.

It some embodiments, that device comprises cells that play a role in the healing and repair process, should that be desired. In some embodiments, the cells may be incorporated into the RTR polymer at a lower temperature, while the system is in its low viscosity state. In some embodiments, the cells may be incorporated into any of the other components of the occluding device.

According to the invention disclosed herein, the occlusion device or occlusion material is delivered into the lumen to be occluded at a temperature below body temperature, at which the device or material is in a liquid or flowable state. Once in the body or in contact with the lumen walls, the temperature of the material or device increases causing gelation and solidification, the speed of which can be fine-tuned by varying the composition of the device. In solidifying in situ, the material or device suitably (physically) attaches to the luminal tissue bed, enabling penetration of the occlusion material into folds, pores and crevices of the luminal tissue, such as the fallopian tubes, and fully conform to it and attaches to it. By doing so, the device fixates at the site due to mechanical interlocking, avoiding its migration over time. That said, seeking to maximize its stability at the luminal site, the occlusion material or device may also be rendered mucoadhesive or tissue adhesive by adding to the RTR aqueous solution a mucoadhesive or tissue adhesive component, water soluble or not, such as polyacrylic acid or chitosan, among others.

The occluding device of the invention can be removed or replaced easily and rapidly by lowering the temperature and liquefying, partially or fully, the device. During liquification, the device disengages from the luminal tissue without traumatizing the luminal tissues and without pain.

These and the other unique features of the invention taught hereby are very advantageous to all patients. Furthermore, these features are of a critical importance and special benefit for patients with specific pathologies such as, without limitation, diabetes and hemophilia, where even a minor bleeding may become a major complication, or in situations where bleeding should be avoided due to the spreading of infectious or other diseases.

Occlusion materials or RTR polymers of the invention may be prepared utilizing synthetic methodologies known in the art from various building blocks. Exemplary processes, provided in non-limiting embodiments, are disclosed herein.

RTR polymers of the invention are tailored as occlusion materials for in vivo instillation. Methods of instillation of the material in a subject's body lumen may be regarded methods of occluding the body lumen. The methods may involve invasive or semi-invasive techniques and may thus be practiced on a sedated or partially sedated subject.

A method of occluding a body lumen is provided, wherein the method comprises:
delivering at least one occlusion material or composition to a region of the lumen to be occluded, the material or composition being as disclosed herein, e.g., having a solid state at a physiological temperature and a liquified or flowable state at a temperature below physiological temperature, wherein delivering is achievable at a temperature below physiological temperature; and
allowing the occlusion material or composition to solidify, thereby occluding the region of the lumen.

As used herein, the lumen to be occluded is any cavity of the human or animal body defined by walls, which occlusion may be desired for achieving a timed medical benefit. The lumen may be any tubular body structure that is lined by a body tissue, such as the large intestine, small intestine, veins, arteries, fallopian tubes, vas deferens, urethra and others. The occlusion may be for a period of time spanning several minutes to several years and in some embodiments maybe permanent. Occluding a lumen means forming a blockage in the lumen to obstruct or block the lumen to an extent that prevents transport, transmission or passage of materials, liquids or solids, and cells e.g., sperm cells, through the blockage.

The blockage or obstruction may be of any size and shape. The dimensions thereof may depend on the lumen, the dimensions of the lumen, the site of obstruction, etc. Typically, the amount of the occluding material and the shape and size of the occluding device may be determined by the practitioner administering the occluding material to the lumen. The blockage may be several millimeters (1 to 10 mm) to several centimeters in length. To achieve effective occlusion, the occlusion material may be administered in steps, whereby each step includes administration of an amount of the material, which may be same or different from an occlusion material administered in a previous step. The delivering of the at least one occlusion material or composition to a region of the lumen to be occluded may be achievable by any known medical delivery method. Without wishing to be bound by a specific delivery method, which may be varied or adapted to any one particular lumen or subject, a delivery system, in a shape or form of, e.g., a catheter may be used. The catheter may be inserted via a working channel of a standard hysteroscope, e.g., having a diameter 2.7 mm. It may have one or more channels for injection of the occlusion material with additional channel(s) for cooling the device, as necessary. The catheter may be provided with an angled/shaped tip to facilitate cannulation of the lumen, e.g., fallopian tube, using hysteroscopic visualization. The material occlusion will be delivered in a pre-determined amount or a limited volume such that appropriate injection volume/pressure is applied.

Following delivery, the occlusion material or composition is allowed to solidify into an in situ generated plug or blockage. Solidification is achievable by allowing the device to heat up or warm up to physiological temperature and/or by actively warming the device, from a temperature below the physiological temperature to the physiological temperature, as may be the case. Warming may be achievable by the natural temperature of the body, or may be achieved by applying external heat by utilizing ultrasound magnetic nanoparticles under an external alternating magnetic field, by flowing a warmed or a heated fluid along the circumferential surface of the catheter, or by any other means.

The physiological temperature is typically the normal core internal temperature of a human or an animal subject being around 37-38° C., measured most accurately via a rectal probe thermometer. As a ‘normal’ body temperature may vary slightly between individuals, the “physiological temperature” is a temperature between 35-38° C. The “temperature that is below the physiological temperature” is a temperature below 38° C. or a temperature below 35° C., or a temperature that is between 30 and 35° C.

The temperature below the physiological temperature may in fact be any temperature that is non-injurious to the subject's body and which is determined by the medical practitioner instilling the occlusion device. As such, the temperature may be between 15 and 35° C., or between 20 and 35° C., or between 25 and 35° C., or between 30 and 35° C.

For a timed occlusion, aimed for the treatment of a particular organ or tissue, occlusion of the large intestine, small intestine, veins, arteries, and the urethra may be possible. For controlling human or animal reproduction either the fallopian tubes or vas deferens may be occluded for long periods of time, e.g., months to years. Full reproductive capabilities may be restored on command or on demand as disclosed herein.

For achieving medical benefits, long or short, the occlusion material may be delivered in combination with at least one drug or at least one active material, as disclosed herein.

In some embodiments, the occluding device of the invention is configured for use in a method of occluding a female's fallopian tube(s) or a man's vas deferens for a period of days to years. The occluding device is further configured to be removed from the subject's body to thereby restore reproductive capabilities. Thus, the invention further provides a method of reversibly occluding a body lumen being a fallopian tube(s) or vas deferens, the method comprising
delivering at least one occlusion material or composition to a region of the fallopian tube(s) or vas deferens, the material or composition having a solid or semi-solid state at a physiological temperature and a liquified or flowable state at a temperature below physiological temperature, wherein delivering is achievable at a temperature below physiological temperature;
allowing the occlusion material or composition to solidify into a solid or semi-solid blockage or plug in said fallopian tube or vas deferens; and
liquifying the solid blockage to permit its full or partial removal from the fallopian tube or vas deferens, if required.

In methods of the invention, the occlusion material or composition is any of the materials or compositions disclosed herein, in any combination.

A method is provided for clearing an occluded body lumen, wherein occlusion is provided by a solid or a semi-solid reverse thermo-responsive (RTR) polymer positioned in the body lumen and having a solid or semi-solid state at a physiological temperature and a liquified or flowable state at a temperature below physiological temperature, wherein the occlusion prevents flow or transfer of materials through the body lumen, the method comprising reducing a temperature at the occluded body lumen to a temperature below physiological temperature to thereby liquefy the RTR polymer, restoring flow of materials through the body lumen.

As used herein, the term “clearing” encompasses actively or passively causing removal of the occlusion material following liquification thereof, e.g., at a temperature below the physiological temperature, from the lumen to thereby establish transfer or flow of materials therethrough, and to reestablish the lumen's normal function. The material may be cleared out by normal passive flow from the lumen, or may be caused to degrade in situ, or may be washed, flushed or removed by suction or other means.

A method is further provided for temporarily occluding a body lumen selected from fallopian tube(s) and vas deferens, the method comprising
delivering at least one occlusion device to a region of the lumen to be occluded, the device is or comprising an occlusion material or a composition in a form of a reverse thermo-responsive (RTR) polymer comprising chain extended poly(ethylene oxide); chain extended poly(propylene oxide); di-blocks of poly(ethylene oxide) and poly(propylene oxide); triblocks of poly(ethylene oxide) and poly(propylene oxide); or mixtures thereof,

wherein the RTR polymer having a solid or semi-solid state at a physiological temperature and a liquified or flowable state at a temperature below physiological temperature, wherein delivering is achievable at a temperature below the physiological temperature;
warming the occlusion material or composition to the physiological temperature or to a temperature above the physiological temperature to solidify the material, thereby occluding the region of the lumen; and
at a time period following occlusion of the region of the lumen, clearing the region of the lumen by cooling the solidified material to a temperature below the physiological temperature to thereby liquefy the solidified material.

The time period following occlusion at which the occlusion device or material is cleared out or removed, may be determined by a medical practitioner. Typically, the time period may be days, weeks, months or years following occlusion.

The subject may be human or non-human mammal for veterinary purposes.

Thus, some of the aspects and embodiments of the invention provide:

A stimulus-responsive occlusion device for occlusion a body lumen in vivo, the device exhibiting a solid or semi-solid state at a physiological temperature and a liquified state at a temperature lower than the physiological temperature, the occlusion device is or comprising at least one reverse thermo-responsive (RTR) polymer or a water-formulation comprising a thermo-responsive polymer.

In some embodiments, in a device according to the invention, the RTR polymer comprises chain extended poly(ethylene oxide); chain extended poly(propylene oxide); di-blocks of poly(ethylene oxide) and poly(propylene oxide); triblocks of poly(ethylene oxide) and poly(propylene oxide); or mixtures thereof.

In some embodiments, in a device according to the invention, the RTR polymer comprises poly(ethylene oxide)-poly(propylene oxide)-poly(ethylene oxide) (PEO-PPO-PEO) triblocks; random or alternating reverse thermo-responsive PEO-PPO block copolymers; N-alkyl substituted acrylamides; poly(ethylene oxide)-polylactic acid copolymers; poly(ethylene oxide)-polycaprolactone copolymers; and/or amphiphilic polymers.

In some embodiments, in a device according to the invention, the RTR polymer comprises poly(ethylene oxide)/poly(propylene oxide)/poly(ethylene oxide) (PEO-PPO-PEO) triblock.

In some embodiments, in a device according to the invention, the RTR polymer having a structure -[E-(BCB)]n, wherein B is polyethylene oxide (PEO), C is polypropylene oxide (PPO), n is an integer designating the number of blocks in the polymer and being 2 or more, and E is a chain extender moiety connecting the triblocks to each other.

In some embodiments, in a device according to the invention, E is derived from a bifunctional material selected di-isocyanates, di-acyl chlorides, di-carboxylic acids and di-anhydrides.

In some embodiments, in a device according to the invention, E is derived from a di-isocyanate.

In some embodiments, in a device according to the invention, E comprises a urethane moiety.

In some embodiments, in a device according to the invention, the di-isocyanate is selected from the group consisting of hexamethylene di-isocyanate (HDI), methylene di-phenyl di-isocyanate (MDI), isophorone diisocyanate, lysine diisocyanate ethyl ester and toluene di-isocyanate.

In some embodiments, in a device according to the invention, the di-isocyanate is HDI.

In some embodiments, in a device according to the invention, the device comprising one or more additional materials or solid components, optionally being a drug or an active material, selected from the group consisting of drugs and drug residues, oligopeptide sequences, growth factors, hormones, materials containing genetic information, cells, contraceptive agents, ion eluting agents, metals or metallic materials, anti-restenosis agents, antibacterial agents, antifungal agents, antimicrobial agents, and antibiotics.

In some embodiments, in a device according to the invention, the body lumen is fallopian tube or vas deferens.

A method is provided for occluding a body lumen, the method comprising
delivering at least one occlusion device to a region of the lumen to be occluded, the device is or comprising an occlusion material or a composition in a form of a reverse thermo-responsive (RTR) polymer having a solid or semi-solid state at a physiological temperature and a liquified or flowable state at a temperature below physiological temperature, wherein delivering is achievable at a temperature below the physiological temperature; and
warming the occlusion material or composition to the physiological temperature or to a temperature above the physiological temperature to solidify the material, thereby occluding the region of the lumen.

In some embodiments, in a method according to the invention, the RTR polymer comprises poly(ethylene oxide)/poly(propylene oxide)poly(ethylene oxide) (PEO-PPO-PEO) triblocks.

In some embodiments, in a method according to the invention, the RTR polymer is of a structure -[E-(BCB)]n, wherein B is polyethylene oxide (PEO), C is polypropylene oxide (PPO), n is an integer designating the number of blocks in the polymer and being 2 or more, and E is a chain extender connecting a triblock to another triblock.

In some embodiments, in a method according to the invention, E comprises a urethane moiety.

In some embodiments, in a method according to the invention, the body lumen is fallopian tube or vas deferens.

In some embodiments, in a method according to the invention, occlusion is for a period of several days to several years.

### BRIEF DESCRIPTION OF THE DRAWINGS

The accompanying drawings, which are incorporated into and form a part of the specification, illustrate several embodiments of the present invention and, together with the description, serve to explain the principles of the invention.

FIG. 1 presents a generic depiction of the PEO-PPO-PEO triblocks.

FIG. 2 presents the shortcomings of the PEO-PPO-PEO F127 triblock.

FIG. 3 depicts synthesis of the PF127 polyether urethane.

FIGS. 4A-B provide FTIR spectra of F127 (A) and PF127 (B).

FIG. 5 provides NMR spectra of the PF polyether urethane formed.

FIG. 6 provides DSC thermograms of F127 and PF127.

FIG. 7 provides viscosity versus temperature plots for 20%/wt F127 and PF127 solutions.

FIG. 8 provide Ti values for F127 and PF127 solutions (left) and viscosity at 37° C., for 20% wt and 25% wt solutions.

FIG. 9 demonstrates the ability of the RTR device to gel in contact with tissue and the liquefy upon cooling.

FIGS. 10A-B show viscosity versus temperature plots of the F88 triblock and the PF88 chain extended polymer at various concentrations.

FIG. 11 depicts the two-step synthesis of a chain extended polymer comprising F127 and different Jeffamine segments.

FIG. 12 shows a fluoroscopic image showing free spillage of contrast material into the peritoneal cavity (thick arrow) after injection via the right fallopian tube (arrow), thereby confirming tubal patency.

FIG. 13 shows a fluoroscopic image demonstrating absence of contrast passage to the peritoneal cavity (arrow) following injection via the right fallopian tube, thereby confirming continued tubal occlusion. The distended uterine horn (reflux of contrast material) is clearly seen (thick arrow).

FIG. 14 provides fluoroscopic image taken following reversal of tubal occlusion in which a patent right fallopian tube is clearly seen following contrast injection (arrow) as is spillage of contrast into the peritoneal cavity (thick arrow), confirming tube patency.

FIGS. 15A-G depict the different stages of the implantation procedure at experiments performed using the chain extended PF88:15% RTR polymer: (A) Uterine horns and fallopian tubes of the rabbit; (B) Direct cannulation with a 24G Venflon catheter; (C) Injection of contrast dye prior to fallopian tube occlusion (D) Fluoroscopy confirmation of tubal patency; (E) Injection of sterile RTR Polymer (PF88 15%); (F) Injection of contrast dye after fallopian tube occlusion; (G) Fluoroscopy confirmation of tubal occlusion.

FIGS. 16A-E depict the different stages of the procedure 14-28 weeks later: (A) Direct cannulation with a 24G Venflon catheter; (B) Injection of contrast dye to test fallopian tube occlusion; (C) Fluoroscopy confirmation of tubal occlusion (D) Cooling the fallopian tube while injecting contrast dye; (E) Fluoroscopy confirmation of tubal patency.

### DETAILED DESCRIPTION OF EMBODIMENTS

For sake of clarity, conciseness and simplicity, and without detracting from the generality of the technology in any form or fashion, the inventors have chosen to illustrate the invention by focusing on its use as an occlusion device suitable for deployment in the luminal cavity of the fallopian tubes, where the environmental stimulus is temperature. It is clear that the technology is equally applicable for occlusion of other lumens, utilizing materials as disclosed herein and stimuli that may be selected to meet a particular material composition or utility.

Some of the embodiments of the invention disclosed hereby will be exemplified using the family of polymers comprising poly(ethylene oxide) (PEO) and poly(propylene oxide) (PPO) chains. These can be part of di or triblocks, such as poly(ethylene oxide)-poly(propylene oxide)-poly(ethylene oxide) (PEO-PPO-PEO) triblocks, commercially available as Pluronic©, {EO}₉₉-{PO}₆₇-{EO}₉₉, and {EO}₁₀₃-{PO}₃₉-{EO}₁₀₃, known as Pluronic F127 and Pluronic F88, respectively, being two leading examples.

Also, high molecular weight RTR polymers produced by covalently binding PEO-PPO-PEO triblocks using reactive bi-functional molecules such a di-isocyanates, di-acyl chlorides, phosgene, among others, were used as well. Among them, hexamethylene diisocyanate (HDI), may be used. Additionally, block polymers consisting of poly(ethylene oxide) (PEO) and poly(propylene oxide) (PPO) segments, coupled via diverse coupling agents, such as di-isocyanates, di-acyl chlorides, di-carboxylic acids, phosgene, among others, were used as well. Other chemistries such as Michael addition, thiol-ene and click chemistry mechanisms may be used, among numerous others, as required.

[a] Synthesis of PF-127. Pluronic F-127 (molecular weight 12,600) was poured in a three-necked flask and dried. Then, the corresponding amount of HDI and SnOct₂ (0.64 wt %) were added to the reaction mixture and reacted at 80° C. for 30 minutes under mechanical stirring (160 RPM) and dry nitrogen atmosphere. The polymer produced was dissolved in chloroform and precipitated in a petroleum ether 40-60 ethyl ether mixture (1:1). Finally, the polymer was washed repeatedly with portions of petroleum ether and dried. Different F-127/HDI ratios resulted in different degrees of polymerization (DP).

[b] Synthesis of Poly(ether-carbonate)s. These polymers were synthesized by copolymerizing poly(ethylene glycol) and poly(propylene glycol) segments utilizing phosgene as the coupling molecule. The different reactivity of phosgene's two functionalities allowed binding the two constituents, in both an alternate or random mode.

The synthesis of the alternating poly(ether-carbonate)s was carried out following a two-step reaction, as described elsewhere in detail. The first step was the PEO dichloroformate synthesis, followed by the reaction between the PEG derivative and the PPG chain, to produce the final block copolymer. The random poy(ether-carbonate)s were synthesized by a similar one-pot reaction, as described elsewhere in detail.

[c] Synthesis of Poy(ether-ester)s. The synthesis is exemplified hereby for a copolymer containing PEO6000 and PPO3000 segments. Equimolar amounts of dry PEG6000 and dry PPG3000 were dissolved in 30 ml dry chloroform in a 250 ml flask.

Triethylamine (2:1 molar ratio to PEG) was added to the reaction mixture, followed by the dropwise addition of the diacyl chloride (2:1 molar ratio to PEG) in dry chloroform over a period of 30 minutes at 40° C., under magnetic stirring. Then, the temperature was risen to 60° C. and the reaction was continued for additional 90 minutes. The polymer produced was separated from the reaction mixture by adding to it about 600 ml petroleum 14 ether 40-60. The lower phase of the two-phase system produced was separated and dried at RT. Finally, the polymer was thoroughly washed with petroleum ether and dried. Light yellow, brittle and water soluble powders were obtained.

[d] Synthesis of Poy(ether-ester-carbonate)s. The synthesis is exemplified hereby for a copolymer containing PEO6000 and PPO3000 segments, caprolactone blocks comprising four repeating units and phosgene. The (CL)₄-PEO6000-(CL)₄ triblock was synthesized as follows: 30.3 g of PEG6000 were dried at 120° C. under vacuum for 2 hours. Then, 10.1 g ε-caprolactone and 0.05 g stannous 2-ethyl-hexanoate were added. The reaction mixture was heated at 145° C. for 2.5 hours in a dry nitrogen atmosphere

Finally, the reaction mixture was cooled to room temperature, dissolved in chloroform, precipitated in petroleum ether and dried at room temperature. Once the (CL)₄-PEO6000-(CL)₄ triblock was obtained, the reaction with phosgene and the final reaction with the PPG chain were performed as described above.

In some instances, also biologically active molecules were added to the systems, by just blending them into one or more components of the occlusion device, including the RTR component. In other instances, it was covalently bound to the RTR component or the substrate, in the case the device comprises one.

Furthermore, an “on command” strategy” is easily implemented, whereby the occlusion device is cooled down in a controlled manner, so to allow the faster release of enhanced doses of the drug, at specific time points, as required clinically.

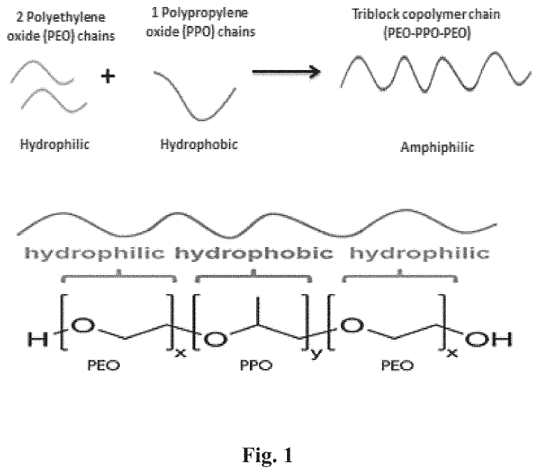

The basic building blocks of the RTR polymers developed are hydroxyl terminated polyethylene oxide-polypropylene oxide-polyethylene oxide (PEO-PPO-PEO) triblocks of various compositions and molecular weights. FIG. 1 present the generic formula of these triblocks. Even though several triblocks were used, most of the work described herein focuses on PEO₉₉-PPO₆₅-PEO₉₉ and PEO₁₀₂-PPO₃₉-PEO₁₀₂ triblocks, named F127 and F88, respectively. The F127 triblock has a molecular weight of 12,600 dalton and a 70% wt PEO content, while F88 has a molecular weight of 11,250 dalton and an 80% wt PEO content.

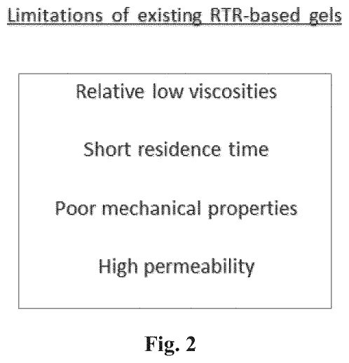

FIG. 2 lists the drawbacks of the existing triblocks. In light of the triblock's significant shortcomings, new RTR polymers displaying improved properties, including triblocks coupled via a suitable chain extender were prepared. As described below, several strategies were pursued seeking to generate occluders able to comply with the especially stringent requirements posed the fallopian tubes blocking plug.

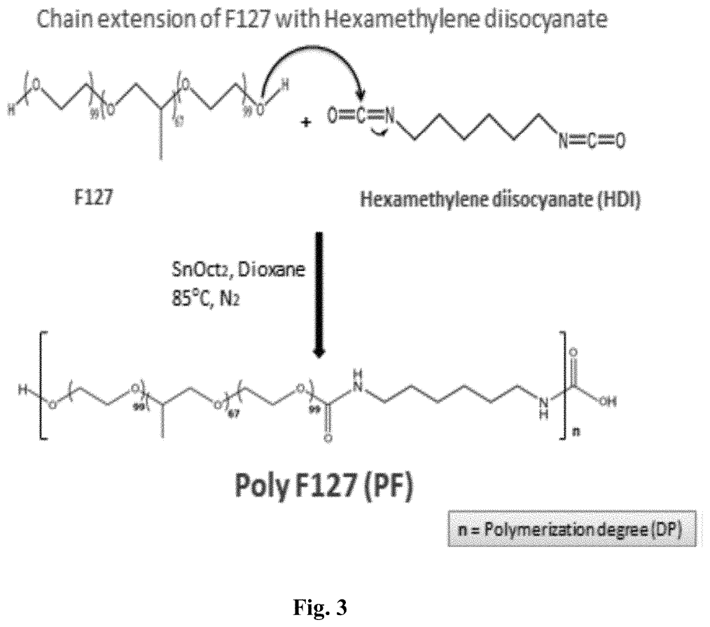

One approach implemented related to the hydroxyl-terminated PEO-PPO-PEO triblocks as a monomeric diol that was then polymerized by reacting it with a chain extender, such as diisocyanates, typically hexamethylene diisocyanate (HDI). While the basic triblocks will be labelled “F”, the high molecular weight polymers are denominated “PF”. The synthesis of the resulting chain extended PF polyether urethanes is shown in FIG. 3.

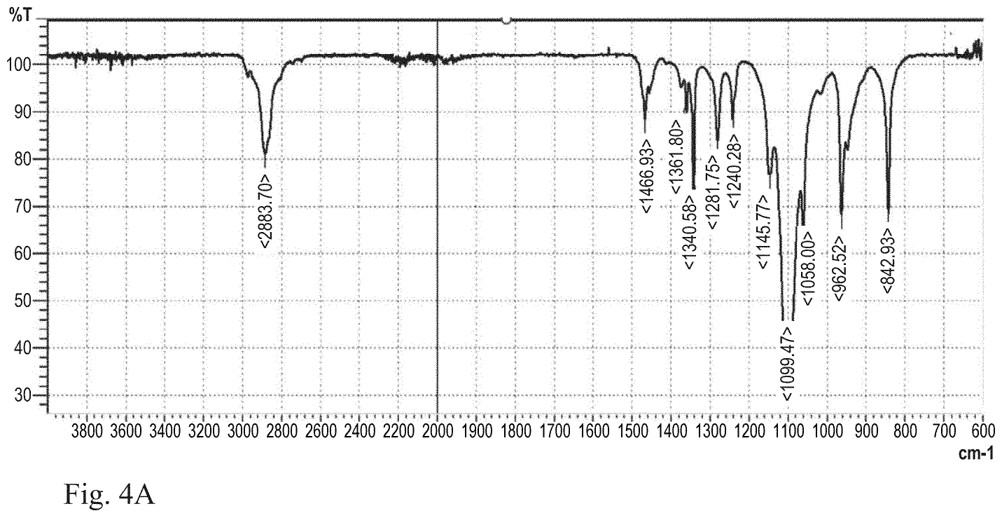

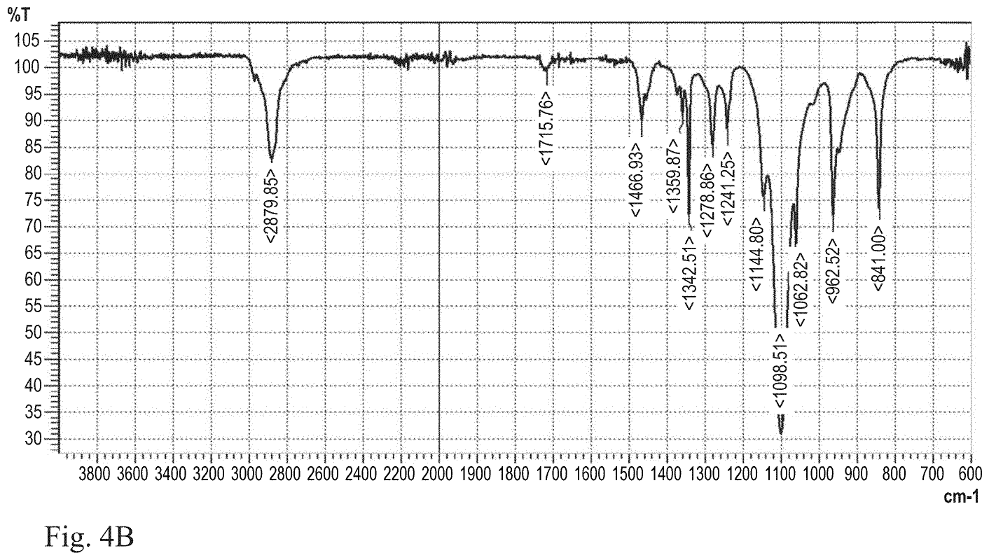

The PF polymers formed were characterized initially by GPC, which demonstrated that the chain extension reaction took place producing polymers having different degrees of polymerization. The fact that actual polyether urethane backbones were generated was also proven by FT-IR and NMR spectroscopies, as exemplified in FIGS. 4A and B for F127 and the resulting PF127 chain extended polymer.

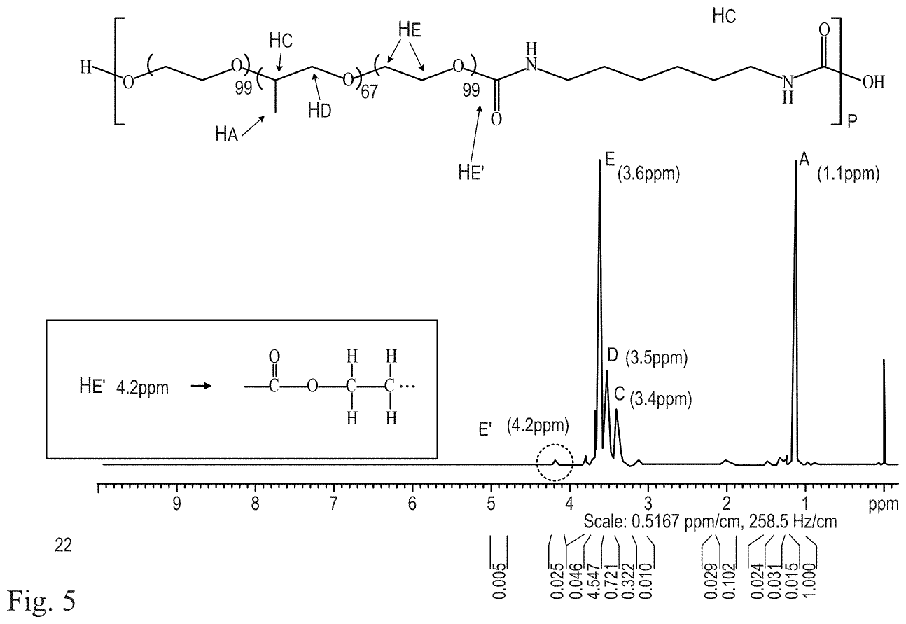

Expectedly, F127 as well as PF127 spectra show very large ether peaks around 1100 cm⁻¹ due to the two hundred and sixty-five ether groups present in the F127 repeating unit. Of particular importance, though, is the peak at 1715 cm⁻¹, absent in F127's spectrum but clearly shown by PF127, due to the urethane carbonyl group formed by the reaction between F127's hydroxyl end groups and HDI's isocyanate moieties. Additional strong evidence of the occurrence of the chain extension reaction was provided by NMR spectroscopy, as presented in FIG. 5. The key finding of the NMR spectrum pertains to the appearance of a new peak at 4.2 ppm. This absorbance band is assigned to the very last ethylene oxide unit present in the PEO segments that is now covalently bound to the just formed urethane group, causing the peak to shift from 3.6 ppm to 4.2 ppm.

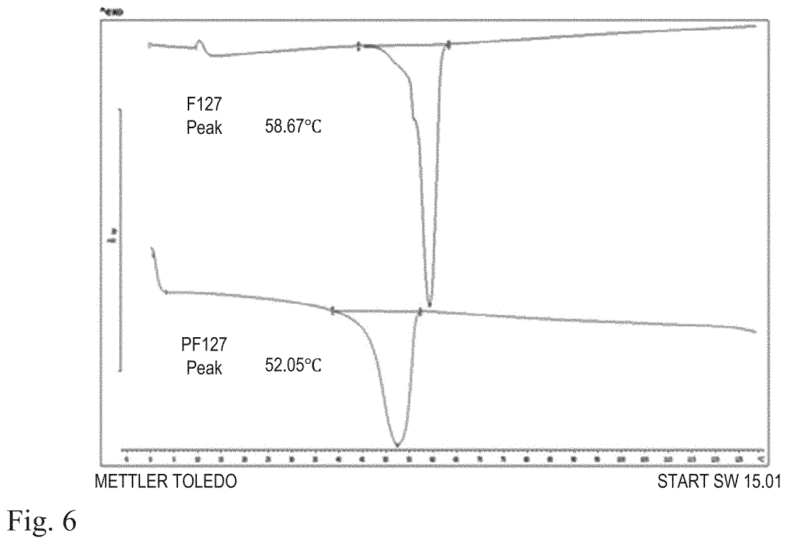

On a morphological level now, the DSC thermograms shown in FIG. 6, show the effect of the polymerization of the PEO-PPO-PEO triblocks on the crystallizability of their semi-crystalline PEO segments. This is revealed by the fact that the melting peak of “monomeric” F127 shifted after the chain extension reaction from at 59° C. to 52° C. in PF127. Furthermore, the sharp melting endotherm shown by F127 has significantly broadened in PF127.

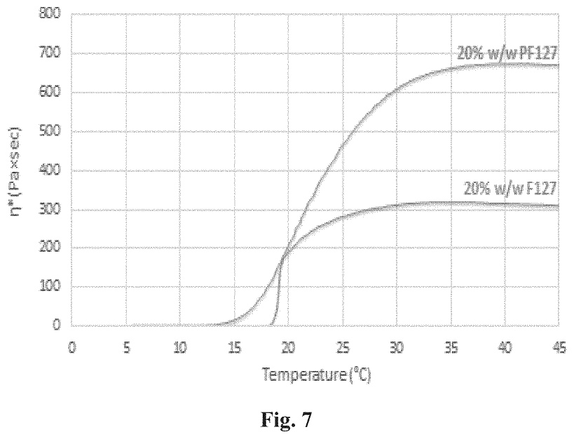

In accordance with theoretical considerations, the markedly larger PF127 chains formed solutions that, once gelled, attained much higher viscosity values, as shown in FIG. 7. It is also worth stressing that the PF127 solution has a lower Ti, the temperature of initiation of gelation, due to its higher molecular weight.

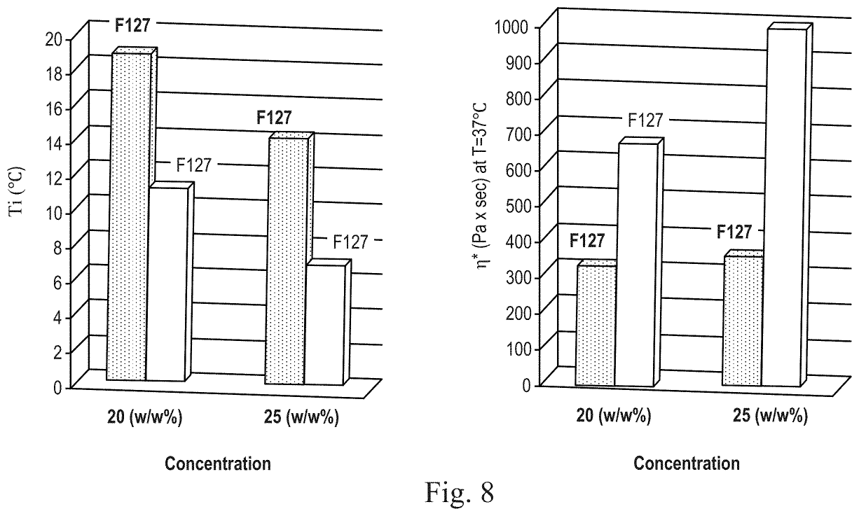

FIG. 8 presents the Ti values for F127 and PF127 solutions and the viscosity they attained at 37° C., for 20% wt and 25% wt solutions. The data showed in FIG. 8 shed light not only on the effect of the length of the chain, F127 versus PF127, on Ti and on the viscosity of the gel at body temperature, but also on the effect of the concentration of the RTR polymer on them.

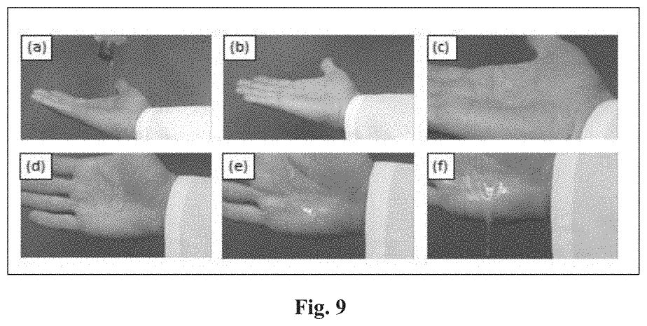

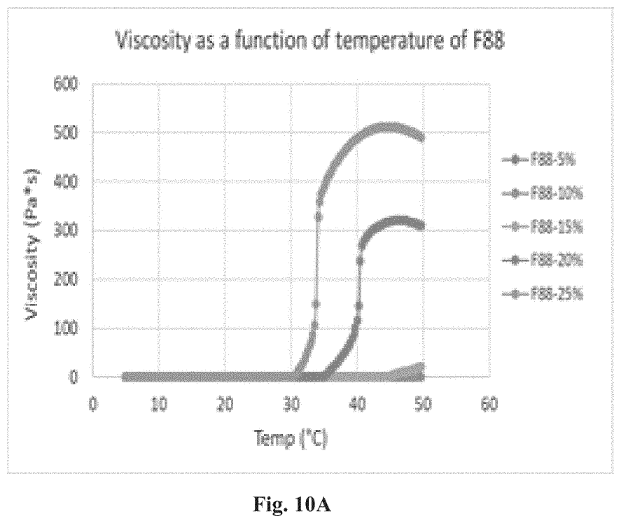

FIGS. 9A-F illustrates the ability of the device to gel in contact with tissue and then liquefy upon cooling, the key feature that will allow the formation of the occlusion device by gelation and its removal by liquefication. Exemplified is an essentially 2D system in contact with a tissue of the hand, followed by its liquefaction by cooling. In this depiction, the RTR occluding formulation is poured (A) on the palm of the hand at a suitable temperature, below its relevant thermal sol-gel transition, so it is a solution, the viscosity of which can be tuned. Once in contact with the skin of the hand, loosely mimicking the luminal organ, the RTR solution heats up (B), crossing its thermal transition and gelling on the tissue (C-D). Subsequently, when required, the occluding gel is cooled down by various means, in this case using a cooling spray (E), whereby the gel is liquefied and easily and more importantly, non-injuriously removed from the site (F).

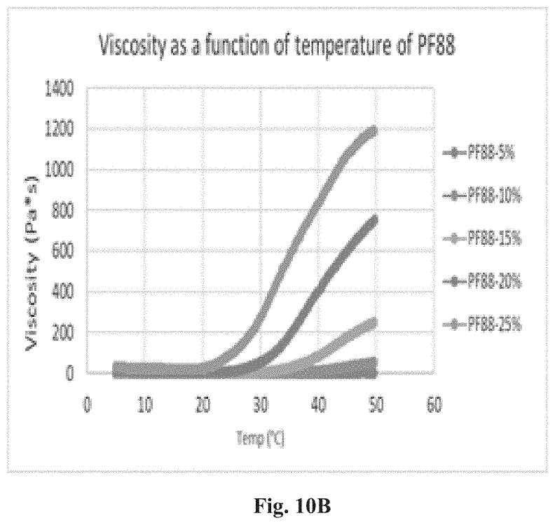

FIG. 10A depicts viscosity versus temperature plots of the F88 triblock, while FIG. 10B presents viscosity versus temperature plot of the PF88 chain extended polymer both at various concentrations. These figures demonstrate the enhanced rheological properties of the chain extended PF88 polymer.

Chain extended polymers comprising other groups, such as urea or amide groups along their backbone were synthesized. In one of the embodiments, urea functional groups were formed by reacting isocyanate and amine moieties.

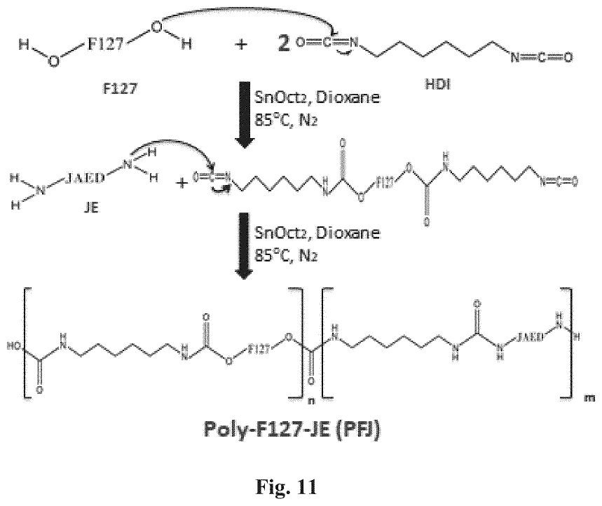

Two synthetic strategies were pursued. In the first one, OH-terminated triblocks, such as F127 and F88, and amine-terminated molecules, such as, for example, Jeffamine chains of various molecular weights were mixed at different molar ratios and randomly chain extended with HDI. Seeking to generate a more ordered polymer expected to exhibit enhanced rheological properties, a two-stage synthetic scheme was followed, as described in FIG. 11. The polymers formed were denominated PFJ.

Initially, the F127 triblock was reacted in a 1:2 molar ratio with HDI, whereby the corresponding macrodiisocyanate was produced, which was subsequently chain extended using various amine-terminated molecules, such as various Jeffamines. Amides groups were generated along the polymeric chain by reacting isocyanate moieties with carboxylic acid groups, for example by oxidizing the hydroxyl end group of the triblocks to a COOH group and then chain extending the triblock with a diisocyanate, for example HDI.

### In Vivo Work:

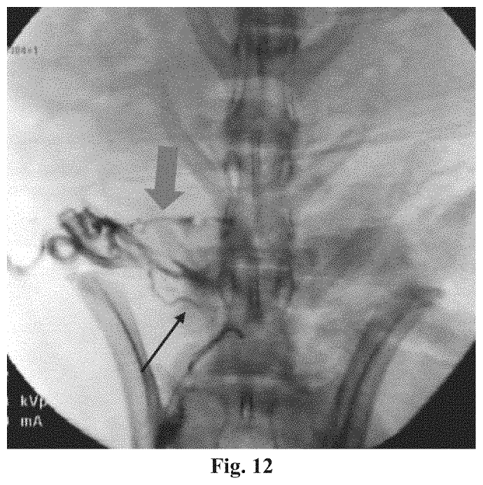

Eight female New Zealand rabbits of approximately -3.5 Kg weight were included in the study protocol. Briefly, under general endotracheal anesthesia the uterine horns and fallopian tubes were surgically exposed and cannulation of the distal uterine using a 24 Gauge Venflon needle catheter as standard, was performed in the direction of the fallopian tube. It should be stressed that all injections into the fallopian tubes were performed during simultaneous temporary occlusion of the uterine horn by external compression, in order to prevent reflux into the uterus. The tissues were maintained at approximately body temperature throughout, by external warming with pre-heated saline soaked gauze. Initially fallopian tubal patency was confirmed by injecting warmed saline and then water soluble iodinated contrast agent to demonstrate spillage into the peritoneal cavity under continuous fluoroscopic control (FIG. 12). The contrast agent was then flushed out using warm normal saline. Tubal occlusion was then performed by injecting ˜0.6 ml of the PF88:15% RTR polymer. Occlusion was confirmed by repeating the injection of warmed iodinated contrast material with fluoroscopic control by demonstrating lack of transit via the fallopian tube into the peritoneal cavity. The animals were then re-awakened and returned to the holding area.

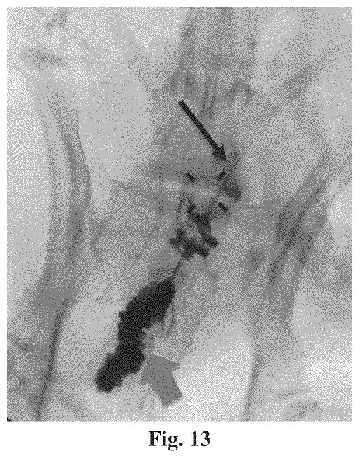

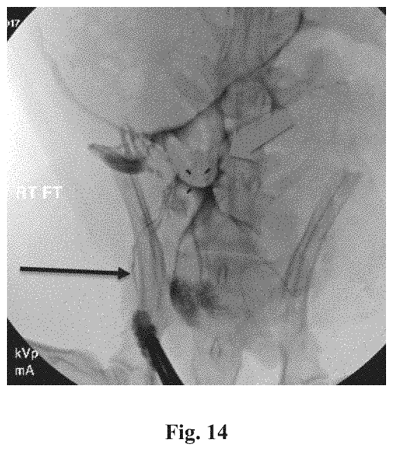

Between 3.5 (n=2) and seven (n=3) months later the animals were returned to the experimental surgery laboratory and again using general endotracheal anesthesia the abdomen was opened surgically and the fallopian tubes/uterine horns were exposed, and maintained at ˜37° C., as described above. Following repeat cannulation of the tubes iodinated contrast material was injected to confirm tubal occlusion under fluoroscopic control (FIG. 13). The fallopian tubes were then individually exposed to iced water for the purpose of liquefying the occlusive polymer agent and achieve recanalization. The warmed contrast agent was once again injected under fluoroscopic control to confirm reversal of occlusion (FIG. 14). The animals were euthanized using standard approved techniques.

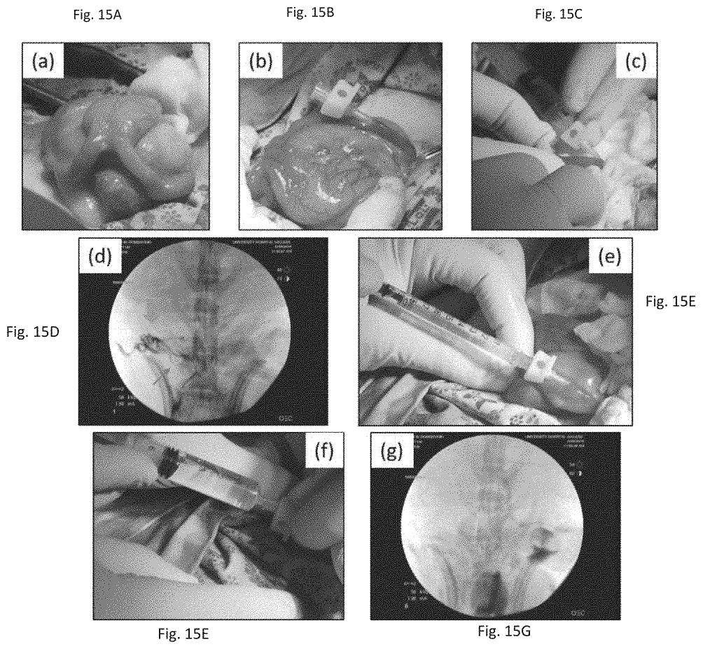

Histological analysis was performed on several specimens obtained after reversal of fallopian tube occlusion. There was no evidence of inflammation, infection or fibrosis is the specimens that were examined. Tube lumen appeared to be within normal limits with maintenance of patency (FIG. 15).

No major adverse events affecting the wellbeing of the animal subjects were encountered during the study period.

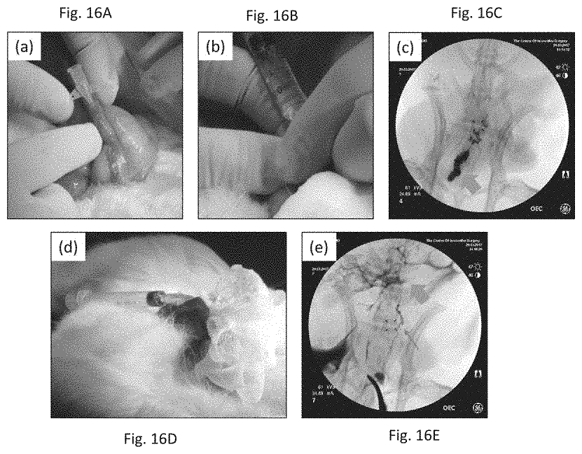

FIG. 16 shows the different stages of the procedure at experiments performed during using the same polymer 14-28 weeks later

1. A stimulus-responsive occlusion device for occlusion a body lumen in vivo, the device exhibiting a solid or semi-solid state at a physiological temperature and a liquified state at a temperature lower than the physiological temperature, the occlusion device is or comprising at least one reverse thermo-responsive (RTR) polymer or a water-formulation comprising a thermo-responsive polymer.

2. The device according to claim 1, wherein the RTR polymer comprises chain extended poly(ethylene oxide); chain extended poly(propylene oxide); di-blocks of poly(ethylene oxide) and poly(propylene oxide); triblocks of poly(ethylene oxide) and poly(propylene oxide); or mixtures thereof.

3. The device according to claim 1, wherein the RTR polymer comprises poly(ethylene oxide)-poly(propylene oxide)-poly(ethylene oxide) (PEO-PPO-PEO) triblocks; random or alternating reverse thermo-responsive PEO-PPO block copolymers; N-alkyl substituted acrylamides; poly(ethylene oxide)-polylactic acid copolymers; poly(ethylene oxide)-polycaprolactone copolymers; and/or amphiphilic polymers.

4. The device according to claim 3, wherein the RTR polymer comprises poly(ethylene oxide)/poly(propylene oxide)/poly(ethylene oxide) (PEO-PPO-PEO) triblock.

5. The device according to claim 4, wherein the RTR polymer having a structure -[E-(BCB)]n, wherein B is polyethylene oxide (PEO), C is polypropylene oxide (PPO), n is an integer designating the number of blocks in the polymer and being 2 or more, and E is a chain extender moiety connecting the triblocks to each other.

6. The device according to claim 5, wherein E is derived from a bifunctional material selected di-isocyanates, di-acyl chlorides, di-carboxylic acids and di-anhydrides.

7. The device according to claim 5, wherein E is derived from a di-isocyanate.

8. The device according to claim 7, wherein E comprises a urethane moiety.

9. The device according to claim 7, wherein the di-isocyanate is selected from the group consisting of hexamethylene di-isocyanate (HDI), methylene di-phenyl di-isocyanate (MDI), isophorone diisocyanate, lysine diisocyanate ethyl ester and toluene di-isocyanate.

10. The device according to claim 9, wherein the di-isocyanate is HDI.

11. The device according to claim 1, comprising one or more additional materials or solid components, optionally being a drug or an active material, selected from the group consisting of drugs and drug residues, oligopeptide sequences, growth factors, hormones, materials containing genetic information, cells, contraceptive agents, ion eluting agents, metals or metallic materials, anti-restenosis agents, antibacterial agents, antifungal agents, antimicrobial agents, and antibiotics.

12. The device according to claim 1, wherein the body lumen is fallopian tube or vas deferens.

13. A method of occluding a body lumen, the method comprising
delivering at least one occlusion device to a region of the lumen to be occluded, the device is or comprising an occlusion material or a composition in a form of a reverse thermo-responsive (RTR) polymer having a solid or semi-solid state at a physiological temperature and a liquified or flowable state at a temperature below physiological temperature, wherein delivering is achievable at a temperature below the physiological temperature; and
warming the occlusion material or composition to the physiological temperature or to a temperature above the physiological temperature to solidify the material, thereby occluding the region of the lumen.

14. The method according to claim 13, wherein the RTR polymer comprises poly(ethylene oxide)/poly(propylene oxide)/poly(ethylene oxide) (PEO-PPO-PEO) triblocks.

15. The method according to claim 13, wherein the RTR polymer is of a structure -[E-(BCB)]n, wherein B is polyethylene oxide (PEO), C is polypropylene oxide (PPO), n is an integer designating the number of blocks in the polymer and being 2 or more, and E is a chain extender connecting a triblock to another triblock.

16. The method according to claim 15, wherein E comprises a urethane moiety.

17. The method according to claim 13, wherein the body lumen is fallopian tube or vas deferens.

18. The method according to claim 13, wherein occlusion is for a period of several days to several years.

19. A method of clearing an occluded body lumen, wherein occlusion is provided by a solid or a semi-solid reverse thermo-responsive (RTR) polymer positioned in the body lumen and having a solid or semi-solid state at a physiological temperature and a liquified or flowable state at a temperature below physiological temperature, wherein the occlusion prevents flow or transfer of materials through the body lumen, the method comprising reducing a temperature at the occluded body lumen to a temperature below physiological temperature to thereby liquefy the RTR polymer, restoring flow of materials through the body lumen.

20. A method of temporarily occluding a body lumen selected from fallopian tube(s) and vas deferens, the method comprising
delivering at least one occlusion device to a region of the lumen to be occluded, the device is or comprising an occlusion material or a composition in a form of a reverse thermo-responsive (RTR) polymer comprising chain extended poly(ethylene oxide); chain extended poly(propylene oxide); di-blocks of poly(ethylene oxide) and poly(propylene oxide); triblocks of poly(ethylene oxide) and poly(propylene oxide); or mixtures thereof,
wherein the RTR polymer having a solid or semi-solid state at a physiological temperature and a liquified or flowable state at a temperature below physiological temperature, wherein delivering is achievable at a temperature below the physiological temperature;
warming the occlusion material or composition to the physiological temperature or to a temperature above the physiological temperature to solidify the material, thereby occluding the region of the lumen; and
at a time period following occlusion of the region of the lumen, clearing the region of the lumen by cooling the solidified material to a temperature below the physiological temperature to liquefying the solidified material.
